(19) United States
(12) Patent Application Publication
(10) Pub. No.: US 2024/0238250 A1
(43) Pub. Date: Jul. 18, 2024

(54) METHOD OF USING PIROMELATINE FOR TREATING PARASOMNIAS ASSOCIATED WITH LOSS OF REM SLEEP ATONIA

(71) Applicant: NEURIM PHARMACEUTICALS (1991) LTD., Tel Aviv (IL)

(72) Inventors: Ran FURMAN, Tel Aviv (IL); Nava ZISAPEL, Tel Aviv (IL)

(73) Assignee: NEURIM PHARMACEUTICALS (1991) LTD., Tel Aviv (IL)

(21) Appl. No.: 18/410,317

(22) Filed: Jan. 11, 2024

Related U.S. Application Data
(60) Provisional application No. 63/479,781, filed on Jan. 13, 2023.

(51) Int. Cl. A61K 31/4045; A61K 45/06; A61P 25/00

(52) U.S. Cl. CPC A61K 31/4045; A61K 45/06; A61P 25/00

(57) ABSTRACT

Methods of treating a subject having parasomnias related to Rapid Eye Movement (REM) sleep and attenuating disease progression into synucleinopathies and Parkinson's disease by administering a formulation comprising an effective amount of piromelatine to the subject. Methods of treating a subject having parasomnias related to Rapid Eye Movement (REM) sleep and attenuating disease progression of parasomnias into chronic post-traumatic stress disorder (PTSD) by administering a formulation comprising of an effective amount of piromelatine to a subject.

### CROSS-REFERENCE TO RELATED APPLICATION

This application claims the priority benefit of U.S. provisional application No. 63/479,781 filed Jan. 13, 2023, the contents of which are incorporated herein in their entireties by reference thereto.

### FIELD

Provided herein is a melatonin MT1/MT2/MT3 and serotonin 5-HT1A and 5-HTID receptors agonist, N-(2-(5-methoxy-1H-indol-3-yl)ethyl)-4-oxo-4H-pyran-2-carboxamide (piromelatine), for the manufacture of a medicament for treating parasomnias that involve loss of muscle atonia during REM sleep, e.g., Rapid Eye Movement (REM) sleep behavior disorder (RBD) and REM sleep without atonia (RSWA) and thereby delay or prevent phenoconversion into synucleinopathies and chronic PTSD.

### BACKGROUND

Rapid eye movement sleep (REM sleep or REMS) is a unique phase of sleep, characterized by wake-like rapid, low voltage desynchronized brain waves, rapid eye movement, high dream propensity and low muscle tone (atonia) which prevents humans to act out of their dreams. It is physiologically different from the other phases of sleep, which are collectively referred to as non-REM sleep (NREM sleep, synchronized sleep). REM and NREM sleep alternate during the night and as the night goes on. REM stages get longer, especially towards the last third of the night.

RBD is a parasomnia characterized by dream-enactment behaviors that emerge during a loss of REM sleep atonia (DSM-5 327.42 and ICSD-3 classification). RBD can be considered idiopathic (isolated; iRBD) or can occur in the context of other disorders (e.g., traumatic brain injuries, pesticides exposure, post-traumatic stress disorder (PTSD), antidepressants intake). In patients with RBD, the characteristic atonia of voluntary muscles in REM sleep is impaired, leading to acting out of dreams, particularly in the last third of the night sleep. Initially, patients start to vocalize, speak and move complexly. As the disorder progresses, patients may yell, fight or jump out of bed, often injuring themselves or their sleeping partner. The disorder occurs in 0.5 percent of the population, mostly males older than 50 years.

Abnormally high RSWA may predict conversion into chronic PTSD and patients in general demonstrate increased prevalence of both RSWA and RBD (Feemster J C et al., J Clin Sleep Med. 2019, 15; 15(2):345-349, Elliott J E et al., Sleep J, 2020, 1-10; Feemster J C et al., Sleep J, 2022, Vol. 45, No. 3). In addition, PTSD patients show a substantial loss of slow oscillation power (<1 Hz), yet increased power for higher frequencies during NREM sleep and increased slow oscillation power over occipital areas during REM sleep. The latter effects positively correlate with nightmare activity (Boer et al SLEEPJ, 2020, 1-9).

Recently, iRBD was recognized as the most reliable prodromal biomarker of synucleinopathies such as Parkinson's disease, Lewy body dementia, or multiple system atrophy (MSA) (Iranzo, Fernandez-Arcos et al. 2014, Postuma, Iranzo et al. 2019, Li and Le 2020). A reduction in the spectral power in the REM sleep alpha band is an early marker of phenoconversion of RBD to synucleopathies (Gong S Y et al., Nature and Science of Sleep 2022:14 407-418). RBD patients have a 45% risk of developing Parkinson's Disease (PD) at 5 years and a 76% risk at 10 years (Tianbai L and Weidong L, Neurosci. Bull, 2020, 36(2): 183-194, Fereshtchnejad S M et al., Mov Disord 2017, 32: 865-873; Postuma R B et al., Brain. 2019, 1; 142(3):744-759). As such, RBD offers an early window for preventive treatments against phenoconversion into synucleopathies.

High levels of inflammatory cytokines (tumor necrosis factor-α (TNF-α), C-reactive protein (CRP)) in serum may also predict phenoconversion in RBD patients (Kim, Lee et al. 2020, Wang, Liu et al. 2022).

Currently there are no approved drugs for the treatment of REM parasomnias (RSWA and RBD). Reported treatment outcomes have largely come from clinical experience or case studies (Jiménez-Jiménez J. Pers. Med. 2021, 11, 1204). Clonazepam (a benzodiazepine) and melatonin are the most-commonly used treatment options. However, the use of clonazepam in elderly patients raises many concerns, including exacerbation of obstructive sleep apnea and cognitive impairment, and therefore more tolerable therapies are needed (Jimenez-Jimenez, Alonso-Navarro et al. 2021). Reports on the effects of melatonin in RBD are relatively rare and mostly inconsistent thus raising doubts about the effect in general (Kunz D. and Mahlberg R. J Sleep Res. 2010 December; 19(4):591-6, Jun J. S> et al., Ann Clin Transl Neurol. 2019 April; 6(4): 716-722; Kim et al. 2019, Gilat M et al., Movement Disorders, Vol. 35, No. 2, 2020; Gilat M. et al., Journal of Neurology 2022, 269:125-148). It has been suggested that timed melatonin treatment may delay phenoconversion into parkinsonian syndrome (Kunz D and Bes F. Neuropsychobiology. 2017; 76(2): 100-104), but evidence is still lacking. In PTSD benzodiazepines or other sedative hypnotic medications, which are causal of increased intrusive and dissociative symptoms over time, should be avoided thereby limiting their use in RBD cases linked to PTSD (Guina J et al., J Psychiatr Pract. 2015, 21:281-303).

There are some conflicting data from isolated case reports suggesting a potential for 5-HTIA receptors agonists (e.g., buspirone, SEP-363856) to either cause or resolve somnambulism (sleepwalking) potentially by affecting REM sleep atonia. (Moses T. E. H. and Javanbakht A. J Clin Psychopharmacol. 2022; 42(1): 96-98; Hopkins et al. Translational Psychiatry 2021; 11:228), deterioration in RBD symptoms with tandospirone (5-HT1A receptors partial agonist) was reported (Takahashi et al., Sleep Med. 2008, 9, 317-319). Serotonergic medications (selective serotonin reuptake inhibitors (SSRIs)) may provoke, worsen or improve RBD symptoms REM sleep without atonia. (Winkelman & James Sleep. 2004; 27:317-321) Takahashi et al., Sleep Med. 2008, 9, 317-319).

There appears to be little or no evidence from published articles, that administration of a melatonergic agonist or serotonin 5HT-1A receptor agonist would affect sleep structure in the aspects disclosed herein, (Arbon et al. J Psychopharmacol. 2015 July; 29(7):764-76; Biard et al J Psychopharmacol. 2015 October; 29(10):1106-11.).

Piromelatine, a dual acting serotonergic 5HT-1A and melatonergic agonist has previously been tested for the treatment of insomnia, and Alzheimer's disease (patent U.S. Pat. No. 7,635,710B2, US9101613m Schneider et al J Prev Alz Dis 2021, which are incorporated by reference in their entireties for all purposes), but not for RBD or its phenoconversion into Parkinson disease.

### SUMMARY OF THE INVENTION

In contrast with the results of the above published papers, the present inventors have surprisingly found that investigational compound, N-(2-(5-methoxy-1H-indol-3-yl)ethyl)-4-oxo-4H-pyran-2-carboxamide (piromelatine), an MT1/MT2/MT3 melatonin receptors and serotonin 5-HT1A and 5-HTID receptors agonist, increases the spectral power in the a band during REM sleep in patients with insomnia aged 20-80 years (Examples 1 and 2) with minor or no changes in other frequencies. Because the slowdown of REM sleep a waves is a biomarker of RBD phenoconversion to synucleopathies (Gong S Y et al., Nature and Science of Sleep 2022: 14 407-418), piromelatine is disclosed herein for use to ameliorate RBD symptoms while preventing or delaying phenoconversion into synucleopathies such as like Parkinson's disease. The present disclosure provides an unexpected method of using piromelatine in subjects having RSWA and RBD to inhibit or reduce risk of phenoconversion into synucleinopathies.

Provided herein are methods for treating a subject having parasomnias related to REM sleep comprising administering a formulation comprising an effective amount of piromelatine to the subject as well as a formulation comprising an effective amount of piromelatine for use in treating a subject having parasomnias related to REM sleep. In some aspects, the subject has REM sleep behavior disorder (RBD). In some aspects, the subject has REM sleep without atonia (RSWA).

Provided herein are methods for delivering a piromelatine to a subject for disease modification resulting in prevention, attenuating disease progression, or reducing risk of phenoconversion into synucleinopathies comprising administering a formulation comprising an effective amount of piromelatine to the subject. The present disclosure also includes a formulation comprising an effective amount of piromelatine for use in disease modification resulting in prevention, attenuating disease progression, or reducing risk of phenoconversion into synucleinopathies.

Provided herein are methods of delivering a piromelatine to a subject for treating RBD or attenuating its progression into synucleinopathies or Parkinson's disease comprising administering a formulation comprising an effective amount of piromelatine to the subject. The present disclosure also includes a formulation comprising an effective amount of piromelatine for use in treating RBD or attenuating its progression into synucleinopathies or Parkinson's disease.

Provided herein are methods of delivering a piromelatine to a subject for attenuation progression of parasomnias related to REM sleep into chronic post-traumatic stress disorder (PTSD) comprising administering a formulation comprising an effective amount of piromelatine to the subject. The present disclosure also includes a formulation comprising an effective amount of piromelatine for use in attenuation progression of parasomnias related to REM sleep into chronic post-traumatic stress disorder (PTSD)

According to any of the aspects described herein a formulation comprising an effective amount of piromelatine may comprise the use of a piromelatine in unit dosage form, each unit dosage comprising 2 to 100 mg of piromelatine, in the manufacture of a medicament for treating parasomnia related to REM sleep atonia, e.g., RSWA and RBD and their associated pathologies.

Provided herein are methods of delivering a piromelatine to a subject for attenuating the progression into RBD-associated synuclein-specific neurodegeneration (synucleinopathies) pathologies.

Provided herein are methods for the amelioration of RBD and RSWA in PTSD patients, thereby reducing the risk of turning the disorder into chronic PTSD.

According to any of the aspects described herein a pharmaceutical composition may be used containing as an active substance a therapeutically effective amount of piromelatine, or a pharmaceutically acceptable salt thereof as well as any stereoisomer, in association with one or more pharmaceutically acceptable diluents, preservatives, solubilizers, emulsifiers, adjuvants, excipients or carriers conventionally used in pharmaceutical and veterinary formulations. The present pharmaceutical formulation can be adopted for administration to humans and/or animals.

These and other aspects of the invention will be apparent upon reference to the following detailed description, claims, aspects, procedures, compounds, and/or compositions and associated background information and references, which are hereby incorporated in their entirety.

### BRIEF DESCRIPTION OF THE DRAWINGS

FIG. 1 shows the Effects of 4-wks treatment with piromelatine (Neu-P11) 20 and 50 mg compared to placebo on % REM sleep alpha power according to thirds of the night.

FIG. 2 shows the Effects of 4-wks treatment with piromelatine (NEU-P11) 20 and 50 mg compared to placebo on NREM power spectrum (% of baseline spectral power density).

### DETAILED DESCRIPTION

While aspects of the subject matter of the present disclosure may be embodied in a variety of forms, the following description is merely intended to disclose some of these forms as specific examples of the subject matter encompassed by the present disclosure.

Accordingly, the subject matter of this disclosure is not intended to be limited to the forms or embodiments so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

The singular forms “a,” “an,” and “the” include plural referents unless the context clearly dictates otherwise.

The term “treating”, or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e., not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e., “comprising,” “consisting,” or “consisting essentially”) provides direct support for replacement to any of the other transition term not specifically used. For example, amending a term from “comprising” to “consisting essentially of” would find direct support due to this definition.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value.

Generally herein, the term “or” includes “and/or.”

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Furthermore, certain compositions, elements, excipients, ingredients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of method steps, active agents, kits, or compositions described with respect to piromelatine or method of treating a certain subject is intended to and does find direct support for embodiments related to compositions, formulations of piromelatine or method of treating a certain subject described in any other part of this disclosure, even if those method steps, active agents, kits, or compositions are not re-listed in the context or section of that embodiment or aspect.

As used herein, the term “overnight” may refer to a period of between about 6 to about 18 hours, e.g., 6, 7, 8, 9, 10, 11, 12, 13, 14, 15, 16, 17, or 18 hours, or any range or amount of time between about 6 hours and about 18 hours.

As used herein “percentage ratio” means a measured value divided by a baseline value multiplied by 100.

Provided herein are novel uses and methods of treatment using piromelatine.

Also as used herein, reference to “a” compound, salt or stereoisomer of piromelatine is intended to encompass “one or more” such compounds, salts or stereoisomers. Furthermore, reference to a “compound”, as in the discussion below of pharmaceutical formulations, is also intended to include a salt or stereoisomer of the compound.

Provided herein is a preparation of compositions containing piromelatine wherein the compositions are useful as medicaments. The pharmaceutical compositions contain as an active substance a therapeutically effective amount of piromelatine or a pharmaceutically acceptable salt thereof as well as any stereoisomer; in association with one or more pharmaceutically acceptable diluents, preservatives, solubilizers, emulsifiers, adjuvant, excipients and carriers conventionally used in pharmaceutical and veterinary formulations. The present pharmaceutical formulations can be adapted for administration to humans and/or animals.

A pharmaceutical formulation according to certain aspects preferably is characterized by at least one of the following features: (i) it is adapted to be administered by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration; (ii) it is in unit dosage form, each unit dosage comprising an amount of piromelatine which is within the range of about 2 mg to 100 mg; or (iii) both (i) and (ii).

The formulations further can be characterized in that they can be administered alone or in combination with or in conjunction with other compounds which are known in the art to be useful for the treatment of RBD or RSWA disorders or prevention of associated pathologies, including, but not limited to, PTSD, Parkinson's disease, Dementia with Lewy bodies and multiple system atrophy (MSA).

In some aspects, the methods may comprise administering a formulation comprising an effective amount of piromelatine to the subject to treat RBD and RSWA where the word “treat” as used herein means to alleviate or cure a disease, disorder or condition or to case at least one symptom of the disease, disorder or condition.

In some aspects, the methods may comprise administering a formulation comprising an effective amount of piromelatine to the subject to attenuate progression into phenoconversion of RBD and RSWA-related pathologies, including PTSD into synucleopathies, Parkinson's disease, Dementia with Lewy bodies and multiple system atrophy (MSA). RBD-related and RSWA-related pathologies refer to diseases that directly or indirectly result in or result from RBD and RSWA, or that, in the lack of a clear cause-and-effect link, their prevalence correlate with the prevalence of RBD or RSWA.

In preferred embodiments, the disease or disorder is one suffered by humans and a formulation comprising an effective amount of piromelatine is administered to humans.

Provided herein is a method for treating a subject having parasomnias related to Rapid Eye Movement (REM) sleep comprising administering a formulation comprising an effective amount of piromelatine to the subject. In some aspects, the subject has Rapid Eye Movement (REM) sleep behavior disorder (RBD). In some aspects, the subject has REM sleep without atonia (RSWA).

Provided herein is a method for attenuating disease phenoconversion of parasomnias related to RBD into synucleinopathies comprising administering a formulation comprising an effective amount of piromelatine to the subject.

Provided herein is a method for treating or attenuating disease progression parasomnias related to RBD into Parkinson's disease comprising administering a formulation comprising an effective amount of piromelatine to the subject.

Provided herein is a method of attenuating disease progression of parasomnias related to Rapid Eye Movement (REM) sleep into chronic post-traumatic stress disorder (PTSD) comprising administering a formulation comprising an effective amount of piromelatine to the subject.

In some aspects, a percentage ratio of a REM sleep alpha power band measured overnight may be increased compared to baseline after administering piromelatine to a subject for about 4 weeks. In some aspects, the percentage ratio of the REM sleep alpha power band measured overnight may be about 101% to about 110%, about 102% to about 108%, about 103% to about 107%, about 104% to about 106%, or about 105% compared to baseline after administering piromelatine to a subject for about 4 weeks. In certain aspects, the percentage ratio of the REM sleep alpha power band measured overnight may be increased by about 106% compared to baseline after administering piromelatine to a subject for about 4 weeks.

In some aspects, a percentage ratio for a REM sleep delta power, theta power, sigma power, beta power, beta1 power, beta2 power, and beta3 power are each independently about 97% to about 105%, about 98% to about 104%, about 99% to about 103%, about 100% to about 102%, or about 101% compared to baseline, as measured overnight, and after administering piromelatine to a subject for about 4 weeks.

In some aspects, a non-REM sleep (NREM) low frequency band between 0.5 to 1.5 Hz may be increased compared to baseline after administering piromelatine to a subject for about 4 weeks. In some aspects, a percentage ratio of an NREM low frequency band between 0.5 to 1.5 Hz may be greater than 100% to about 120% or less, about 105% to about 115%, about 107% to about 112%, or about 110% compared to baseline after administering piromelatine to a subject for about 4 weeks.

In some aspects, a formulation comprising an effective amount of piromelatine can be administered alone or in combination for example, as a combination therapy, with other agents known to be beneficial in treating the disease, disorder or condition to be treated. As used herein, “in combination” or “combination therapy” means that piromelatine and the other agent can be co-administered, either in concomitant therapy or in a fixed physical combination, or they may be administered at separate times but so as to complement one another. In some aspects, the formulation comprising an effective amount of piromelatine may be administered in combination with one or more additional therapeutic agents.

In the treatment or prevention of the foregoing conditions, broadly defined as REM disorders, the compound can be administered alone or in combination with other compounds with therapeutic properties, e.g., anti-inflammatory, antioxidant, hypnotics, anxiolytics, antipsychotics, antianxiety agents, minor tranquilizers, melatonin agonists and antagonists, melatonin, benzodiazepines, barbiturates, 5-HT2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, eszopiclone, ethchlorvynol, etomidate, fenobam, flunitrazepam, flurazepam, fluvoxamine, fluoxetine, fosazepam, gaboxadol, glutethimide, halazepam, hydroxyzine, imipramine, indiplon, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, ramelteon, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, valproate, venlafaxine, zaleplon, zolazepam, zolpidem, zopiclone and salts thereof, and combinations thereof, and the like.

Combinations of one or more of these known therapeutic agents with piromelatine will provide additional, complementary, and often synergistic effects to enhance the desirable properties of the known therapeutic agent.

In some aspects, the formulation comprising an effective amount of piromelatine is administered as a combination therapy with other compounds with therapeutic properties, e.g., the compounds listed above. In some aspects, the combination therapy may be administered concomitantly. In some aspects, the combination therapy may comprise separately administering the formulation comprising an effective amount of piromelatine and one or more additional therapeutic agents. In some aspects, the combination therapy may be administered as an admixture.

In some aspects, the combination therapy may comprise an anti-inflammatory, an antioxidant, a hypnotic, an anxiolytic, an antipsychotic, an antianxiety agent, a minor tranquilizer, a melatonin agonist, a melatonin antagonist, melatonin, a benzodiazepine, a barbiturate, a 5-HT2 antagonist, or a combination thereof.

Piromelatine, alone or in combination with one of the aforementioned known therapeutic agents further can be administered in combination with physical treatment methods, such as light therapy, psychiatric therapy etc. In some aspects, the formulation comprising an effective amount of piromelatine may be administered in combination with a physical treatment method. In some aspects, the combination therapy may be administered in combination with a physical treatment method. In some aspects, the physical treatment method may comprise a light therapy, a psychiatric therapy, or a combination thereof.

Piromelatine can be formulated into pharmaceutical composition suitable for oral, parenteral (e.g., intramuscular, intraperitoneal, intravenous or subcutaneous injection, or implant) nasal, vaginal, rectal, sublingual or topical routes of administration. The compositions can comprise one or more pharmaceutically acceptable diluents, preservatives, solubilizers, emulsifiers, adjuvants, excipients and/or carriers. In some aspects, the formulation comprising an effective amount of piromelatine may be administered orally, parenterally, nasally, vaginally, rectally, sublingually, or topically. In some aspects, the formulation comprising an effective amount of piromelatine may be administered in a unit dosage form.

Solid dosage forms for oral administration include capsules, tablets, pills, minitablets, pellets, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the dosage unit form is a capsule, it may contain, in addition to materials of the above type, a liquid carrier such as a fatty oil. Various other materials can be present as coatings or to otherwise modify the physical form of the dosage unit. Tablets and pills can additionally be prepared with enteric coatings and tablets may be coated with shellac, sugar or both.

Liquid dosage forms for oral administration include pharmaceutically acceptable emulsions, solutions, suspensions, Self-microemulsifying drug delivery system (SMEDDS), liposomes, syrups and elixirs containing inert diluents commonly used in the art, such as water. Besides such inert diluents, compositions can also include adjuvants, such as wetting agents, emulsifying and suspending agents, and sweetening, flavoring, and perfuming agents, oils, surfactants and co-surfactants. A syrup or elixir may contain the active compound, sucrose as a sweetening agent, methyl and propyl parabens as preservatives, a dye and a flavoring such as cherry or orange flavor.

Formulations in the form of an orally administered SMEDDS will increase the bioavailability of piromelatine and/or a second agent. The formulation generally includes an emulsion including an oil or lipid material, a surfactant, and a hydrophilic co-surfactant. A poorly water-soluble drug or pharmaceutical is emulsified in the self-microemulsifying excipient formulation thereby increasing the in vivo bioavailability of the drug or pharmaceutical formulation. Additionally, poorly water-soluble drugs, can then be used in combination with piromelatine.

In some aspects, SMEDDS pharmaceutical compositions may be formulated for oral administration of piromelatine, e.g., emulsions, aqueous or oily suspensions, filled hard or soft capsules or syrups or prepared powders or granules, troches, tablets or lozenges. Pharmaceutically acceptable carriers or additives to the formulation include binders such as lactose, saccharose, sorbitol, mannitol, starch, amylopectin, cellulose or gelatin; Excipients such as dicalcium phosphate; Disintegrants such as corn starch or sweet potato starch; Glidants such as magnesium stearate, calcium stearate, sodium stearyl fumarate may be contained

In some aspects, parenteral administration may include sterile aqueous or non-aqueous solutions, suspensions, or emulsions. Sterile compositions for injection can be formulated according to conventional pharmaceutical practice by dissolving or suspending the active substance in a vehicle such as water for injection, a naturally occurring vegetable oil like sesame oil, coconut oil, peanut oil, cottonseed oil, etc., or a synthetic fatty vehicle like ethyl oleate or the like. Buffers, preservatives, antioxidants and the like may be incorporated as required. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions that can be dissolved in sterile water, or some other sterile injectable medium immediately before use.

Compositions for rectal or vaginal administration are preferably suppositories that may contain, in addition to the active substance, excipients such as cocoa butter or a suppository wax. Compositions for nasal or sublingual administration are also prepared with standard excipients well known in the art.

The dosage of active agent in compositions of any of the aspects described herein may vary, provided that a therapeutic amount is administered. Desirably the active agent is administered to a subject (human or veterinary use) in need of such treatment in dosages that will provide optimal pharmaceutical efficacy. The selected dosage depends upon the nature and severity of the disease or disorder to be treated, desired therapeutic effect, the route of administration, and the duration of treatment. Dosage amount also can vary depending on the weight of the patient, and other factors. For example, the dose of piromelatine that induces a change in RBD may be different than the dose required to prevent synucleinopathies. The dose will vary from patient to patient depending on the nature and severity of the disease, the patient's weight, special diets then being followed by the patient, concurrent medication, the bioavailability of the compound upon administration and other factors which those skilled in the art will recognize.

In the treatment of a condition in accordance with certain aspects described herein, an appropriate daily dosage level will generally be about 2-100 mg. The daily dosage amount can be administered in single or multiple doses per day. Preferably, the dosage level will be about 5-50 mg; more preferably about 20-40 mg. According to one or more of the aspects described here, an effective amount of piromelatine may be about 2 mg to about 100 mg, about 5 mg to about 50 mg, or about 20 mg to about 40 mg, or about 30 mg. In some aspects, the effective amount of piromelatine may be about 50 mg.

Formulations according to certain aspects described herein may be in the form of immediate release, or, where appropriate, such as solid formulations for oral administration, can be in extended release forms. Extended release formulations include delayed-, sustained-, pulsed- or controlled-release formulations. Suitable extended release formulations useful for purposes of certain aspects described herein may include the types of formulations described in U.S. Pat. Nos. 6,106,864; 7,053,122; and 7,118,762, incorporated herein by reference in their entirety. Details of other types of suitable release technologies, such as high energy dispersions and osmotic and coated particles can be found, for example, in Verma, R. and S. Garg, Pharmaceutical Technology On-Line, 25(2), 1-14 (2001), also incorporated herein by reference.

In some aspects, a formulation comprising an effective amount of piromelatine may be administered about 10 minutes to about 60 minutes, about 20 minutes to about 40 minutes prior to sleep time, or about 30 minutes prior to sleep time.

The period of time in which an extended release formulation releases the compound varies based upon the indication and the target therapeutic levels. For RBD, for example, it is desirable to limit the pharmacological effects of the compound administered to night-time, e.g., about 8 hours.

Provided herein is a method for treating a subject having parasomnias related to RBD comprising administering a formulation comprising about 2 mg to about 100 mg, about 5 mg to about 50 mg, or about 20 mg to about 40 mg of piromelatine. In some aspects, the subject is diagnosed with RBD by video polysomnography (vPSG). In some aspects, the vPSG measurement is quantified using a RBD Severity Scale (RBDSS). In some aspects, the subject does not have untreated obstructive sleep apnea (OSA). In some aspects, the subject is not taking a selective serotonin reuptake inhibitor (SSRI), a benzodiazepine hypnotic, a Cyp3A inhibitor, melatonin, a beta blocker, or a combination thereof.

In some aspects, the subject is assessed using a dopamine transporter imaging-DaTscan, a blood testing, or a combination thereof. In some aspects, the blood test comprises measuring TNF-α and CRP levels in a serum sample of the subject.

In some aspects, the subject is assessed using a Pittsburgh Sleep Quality Index (PSQI), an Epworth Sleepiness Scale (ESS), or a combination thereof.

In some aspects, the formulation is a pharmaceutical formulation. In some aspects, the pharmaceutical formulation comprises one or more pharmaceutically acceptable diluents, preservatives, solubilizers, emulsifiers, adjuvants, excipients and/or carriers. In some aspects, the formulation is administered orally, parenterally, nasally, vaginally, rectally, sublingually, or topically.

### EXAMPLES

The following examples are intended to exemplify the present disclosures and are not limitations of the claimed invention. All molecules, compositions, methods, assays, and results disclosed in the examples form non-limiting parts of the present disclosure.

### Example 1 Effects of 4-Wks Treatment with Piromelatine 20 and 50 mg Compared to Placebo on REM Sleep Spectral Power

REM sleep EEG power was calculated to evaluate the effects of chronic (4-week) administration of 20 mg and 50 mg piromelatine compared to those of placebo in patients with primary insomnia. Sleep EEG was recorded in 9 different centers in the frame of a 3-arm parallel group study with a random allocation of patients to one of the three treatment group (i.e., of the 121 patients who had exploitable sleep EEG data, 42 received placebo, 39 received piromelatine 20 mg, and 40 received piromelatine 50 mg). The REM EEG spectral profile of the three groups was comparable at baseline (readings done at two consecutive nights prior to treatment) and the treatment effects could be confidently compared as percentage of a baseline value.

Spectral power was calculated for all-night) and REM sleep for the total EEG spectrum [0.5; 32] Hz and in the following frequency bands: delta [0.5; 4] Hz, theta [4; 8] Hz, alpha [8; 13] Hz, sigma [11.5; 15.5] Hz, beta [15.5; 32] Hz, beta1 [15.5; 20.5] Hz, beta2 [20.5; 25.5] Hz, and beta3 [25.5; 32] Hz.

TABLE 1
4-wks treatment effects of piromelatine on REM EEG spectral power (% of
baseline values and range for the whole night sleep) by frequency band

| | Placebo | Piromelatine | Piromelatine |
|---|---|---|---|
| Frequency Band | (n = 42) | 20 mg (n = 39) | 50 mg (n = 40) |
| Total EEG power | 102.18 | 99.76 | 105.33 |
| ([0.5; 32] Hz) | (15.4-216.2) | (63.2-209.3) | (35.5-197.6+) |
| Delta power | 102.36 | 100.29 | 103.73 |
| ([0.5; 4] Hz) | (17.5-183.9) | (37.7-244.2) | (30.9-234.1) |
| Theta power | 99.66 | 101.45 | 105.03 |
| ([4; 8] Hz) | (27.8-282) | (62.7-343) | (63.1-208.3) |
| Alpha power | 101.33 | 103.36 | 106.55* |
| ([8; 13] Hz) | (69.4-220.3) | (58.5-204.1) | (61.4-326.5) |
| Sigma power | 99.06 | 100.34 | 103.14 |
| ([11.5; 15.5] Hz) | (51.6-201) | (57.7-306) | (39.3-191.1) |
| Beta power | 100.3 | 98.95 | 99.55 |
| ([15.5; 32] Hz) | (27.3-270.1) | (50.3-891.1) | (16.9-997.2) |
| Beta1 power | 98.31 | 100.36 | 98.19 |
| ([15.5; 20.5] Hz) | (50.8-243.5) | (53-937.7) | (23.4-1318.4) |
| Beta2 power | 100.46 | 98.51 | 99.93 |
| ([20.5; 25.5] Hz) | (45.4-300.6) | (54.9-828.5) | (15.7-769.9) |
| Beta3 power | 100.97 | 97.56 | 97.73 |
| ([25.5; 32] Hz) | (11.8-491.5) | (40.2-851.8) | (9.5-241.9) |

*Significant difference (P < 0.05) compared to placebo.

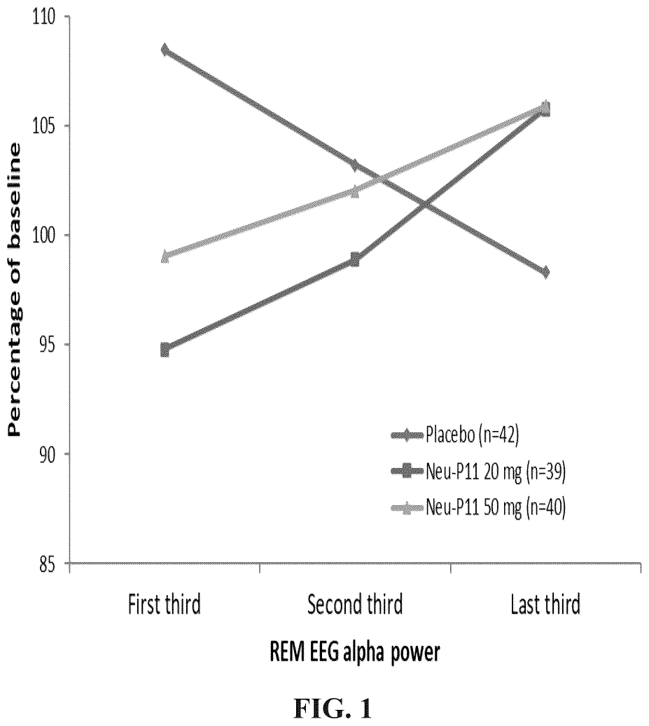

FIG. 1 shows the Effects of 4-wks treatment with piromelatine (Neu-P11) 20 and 50 mg compared to placebo on % REM sleep alpha power according to thirds of the night. Note, REM sleep is longest during the third part of the night.

The results show that piromelatine treatment enhances the REM sleep alpha power band (Table 1) in particular during the longest REM period in the third part of the night sleep (FIG. 1) compared to baseline and to placebo.

### Example 2 Effects of 4-Wks Treatment with Piromelatine 20 and 50 mg Compared to Placebo on NREM Sleep Spectral Power by

NREM sleep EEG power was calculated to evaluate the effects of chronic (4-week) administration of 20 mg and 50 mg piromelatine compared to those of placebo in patients with primary insomnia. Sleep EEG was recorded in 9 different centers in the frame of a 3-arm parallel group study with a random allocation of patients to one of the three treatment group (i.e., of the 121 patients who had exploitable sleep EEG data, 42 received placebo, 39 received piromelatine 20 mg, and 40 received piromelatine 50 mg). The NREM EEG spectral profile of the three groups was comparable at baseline and the treatment effects could be confidently compared as percentage of a baseline value.

Spectral power was calculated for all-night) and NREM sleep for the total EEG spectrum [0.5; 32] Hz and in the following frequency bands: delta [0.5; 4] Hz, theta [4; 8] Hz, alpha [8; 13] Hz, sigma [11.5; 15.5] Hz, beta [15.5; 32] Hz, beta1 [15.5; 20.5] Hz, beta2 [20.5; 25.5] Hz, and beta3 [25.5; 32] Hz.

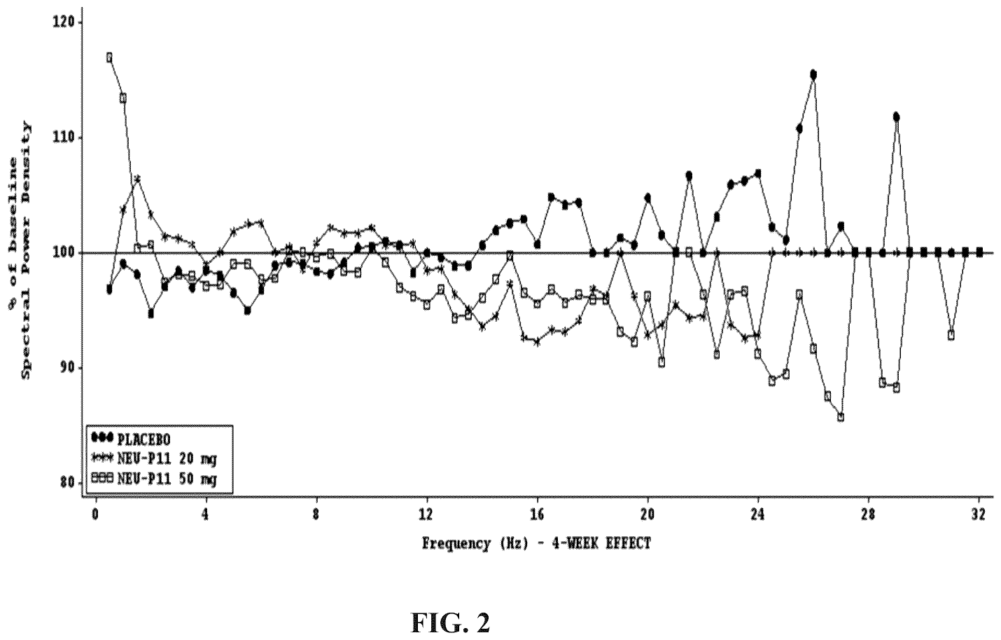

FIG. 2 shows the Effects of 4-wks treatment with piromelatine (NEU-P11) 20 and 50 mg compared to placebo on NREM power spectrum (% of baseline spectral power density). The results show that piromelatine treatment can increase the NREM low frequency band (0.5-1.5 Hz) compared to baseline and to placebo

### Example 3 a Protocol for an Open Label Sleep Laboratory Study of Efficacy and Safety of Piromelatine in a Well-Defines Population of Patients with Idiopathic RBD

### Study Medication:

Piromelatine, 2×20 mg tablets, oral, once daily, at the same clock time (established at 30 minutes prior to patient's habitual bedtime) after a meal, preferably between 21:00 and 23:00.

### Study Aim:

A) To assess the effects of short-term (1 month) treatment with Piromelatine as compared to baseline measures on sleep and RBD symptoms severity.
B) If found efficacious in the short term, to assess the effects of long-term (up to year) treatment with Piromelatine as compared to baseline measures.

### Study Design:

This is proof of concept open label study run in line with recent recommendations for the RBD drug research (Videnovic, Ju et al. 2020). The study protocol will be approved by the ethics committee of the Freie Universitat Berlin. All participants will be asked to provide written, informed consent.

Study participants for clinical trials in RBD should have RBD based on the criteria established by the International Classification of Sleep Disorders (ICSD-3)(ICSD-3 and Medicine 2014), or the Diagnostic and Statistical Manual of Mental Disorders, Fifth edition (DSM-5)(DSM-5 and Association. 2013). Critically, RBD diagnosis requires confirmation by video polysomnography (vPSG), as there are many mimics.

Male or female adult patients aged 50-70 years suffering from RBD as indicated by RBD Single Question Screen (Postuma, Iranzo et al. 2019) and vPSG to confirm that they fulfil criteria for RBD will be included. Up to approximately 12 eligible patients with iRBD (confirmed by vPSG) will be recruited in 6 months.

The Individuals with untreated or sub-optimally treated obstructive sleep apnea (OSA) or other sleep disorders should be excluded. Considering that treatment with antidepressants (SSRIs) may worsen or provoke RBD symptoms, such drugs and benzodiazepine hypnotics should be withdrawn before inclusion. Use of Cyp3A inhibitors (Table 1), melatonin and beta blockers is forbidden in the study. Other drugs must be stable dose for 3 months.

All eligible patients will then undergo vPSG recordings of RBD severity at baseline (3 nights). Due to high night-to-night variation in RBD symptom severity, two nights of vPSG (night −2 and −1) will be performed to reduce variance whereas the first night (night −3) will serve as a habituation night to reduce any effect of sleeping in unfamiliar surroundings on RBD behaviors and will not be analyzed). The vPSG measurement will be quantified using the RBD Severity Scale (RBDSS) that is a validated method to assess both frequency and severity of motor and vocalization events during REM sleep.

Prior to initiation of drug treatment all patients in study will undergo Dopamine transporter imaging—DaTscan, blood testing for general safety and TNF-α and CRP levels in serum. In addition, subjects will fill in the PSQI and ESS.

Subjects will then be given piromelatine and instructed to take the tablet always at the same clock time once per day for 30 days. This clock time should be established at 30 minutes prior to patient's habitual bedtime preferably between 21:00 and 23:00 and after meal. Two nights of vPSG (nights 29 and 30) will be performed. Subjects will then be released until evaluation of the effects of treatment on the severity of their iRBD. If improved, subjects will be allowed to continue into the long-term extension phase (1 year) at which vPSG measurement, DaTscan, serum TNFα and CRP and clinical assessment will be performed. An interim visit/phone call after each month to check safety and clinical status will be performed (Table 2). Patients who have not responded by 1 month will not continue into the long term phase.

### Statistical Analysis:

Demographic data and medical and surgical history will be summarized using descriptive statistics. No formal statistical testing will be performed on these data.

Baseline and end of treatment scores will be summarized at baseline (mean of nights −2-−1), and by the end of the short term 6 weeks treatment (mean of nights 29, 30) or long term (1 year) treatment (days 364, 365) (actual and change from baseline) will be summarized using descriptive statistics (n, mean, SD, median, minimum and maximum). At each visit the mean of the 2 PSG nights will be used in the summaries. Baseline and end of treatment scores (short term, long term) will be compared by paired t-tests. No Bonferroni corrections for multiple comparisons will be performed due to the exploratory nature of the study.

Descriptive statistics will be provided for AEs, physical examination parameters, laboratory parameters and vital signs. The number of patients taking concomitant medications during the treatment period will be summarized by WHO drug classification code for each treatment group.

The number of patients reporting AEs will be summarized by system organ class and preferred term for each treatment group.

The change in laboratory parameters from baseline to the end of the 1 year treatment period will be summarized using shift tables showing the number of patients having values below, within, and above the normal range at each assessment. Data will not be carried forward for patients withdrawing due to treatment failure.

The change in physical examination parameters from baseline to the end of the short term and long term treatment periods will be summarized as the number of patients who have a normal or abnormal examination at each assessment. Data will not be carried forward for patients withdrawing due to treatment failure.

Vital signs will be summarized at baseline and at the end of each treatment period (actual and change from baseline) using descriptive statistics (n, mean, SD, median, minimum, and maximum).

The number of patients withdrawing during each treatment period will be summarized by primary reason for withdrawal. Protocol amendments will be introduced according to ethical and regulatory requirements and subject to approval by the appropriate health authorities.

### Numbered Aspects

The following numbered aspects also form part of the instant disclosure.
1. A method, use, or composition for use for treating a subject having parasomnias related to Rapid Eye Movement (REM) sleep comprising administering a formulation comprising an effective amount of piromelatine to the subject.
2. The method, use, or composition for use of aspect 1, wherein the subject has Rapid Eye Movement (REM) sleep behavior disorder (RBD).
3. The method, use, or composition for use of aspect 1, wherein the subject has REM sleep without atonia (RSWA).
4. A method, use, or composition for use for treating a subject to attenuate disease progression into synucleinopathies comprising administering a formulation comprising an effective amount of piromelatine to the subject.
5. A method, use, or composition for use for treating a subject to attenuate disease progression into Parkinson's disease comprising administering a formulation comprising an effective amount of piromelatine to the subject.
6. A method, use, or composition for use for attenuating disease progression of parasomnias related to Rapid Eye Movement (REM) sleep into chronic post-traumatic stress disorder (PTSD) in a subject comprising administering a formulation comprising an effective amount of piromelatine to the subject.
7. The method, use, or composition for use of any one of aspects 1-6, wherein a non-REM sleep (NREM) low frequency band between 0.5 to 1.5 Hz is increased compared to baseline after administering piromelatine to the subject for about 4 weeks.
8. The method, use, or composition for use of aspect 7, wherein a percentage ratio of an NREM low frequency band between 0.5 to 1.5 Hz is greater than 100% to about 120% or less compared to baseline after administering piromelatine to the subject for about 4 weeks.
9. The method, use, or composition for use of any one of aspects 1-8, wherein the effective amount of piromelatine is about 2 mg to about 100 mg per day, about 5 mg to about 50 mg per day, or about 20 mg to about 40 mg per day.
10. The method, use, or composition for use of any one of aspects 1-9, wherein the effective amount of piromelatine is about 50 mg per day.
11. The method, use, or composition for use of any one of aspects 1-10, wherein the formulation comprising an effective amount of piromelatine is administered orally, parenterally, nasally, vaginally, rectally, sublingually, or topically.
12. The method, use, or composition for use of any one of aspects 1 to 11, wherein the formulation comprising an effective amount of piromelatine is administered in a self-microemulsifying drug delivery system (SMEDDS).
13. The method, use, or composition for use of any one of aspects 1 to 12, wherein the formulation comprising an effective amount of piromelatine is administered in a unit dosage form.
14. The method, use, or composition for use of any one of aspects 1-13, wherein the formulation comprising an effective amount of piromelatine is administered once daily.
15. The method, use, or composition for use of any one of aspects 1-14, wherein the formulation comprising an effective amount of piromelatine is administered about 10 minutes to about 60 minutes prior to sleep time, or about 30 minutes prior to sleep time.
16. The method, use, or composition for use of any one of aspects 1-15, wherein the formulation comprising an effective amount of piromelatine is administered orally.
17. The method, use, or composition for use of any one of aspects 1-16, wherein the formulation comprising an effective amount of piromelatine is administered as a combination therapy with a second therapeutic agent.
18. The method, use, or composition for use of aspect 17, wherein the combination therapy is administered concomitantly with the second therapeutic agent.
19. The method, use, or composition for use of aspect 17, wherein the combination therapy is administered as an admixture with the second therapeutic agent.
20. The method, use, or composition for use of aspect 17, wherein the combination therapy comprises separately administering the formulation comprising an effective amount of piromelatine and one or more additional therapeutic agents.
21. The method, use, or composition for use of any one of aspects 17-20, wherein the combination therapy comprises an anti-inflammatory, an antioxidant, a hypnotic, an anxiolytic, an antipsychotic, an antianxiety agent, a minor tranquilizer, a melatonin agonist, a melatonin antagonist, melatonin, a benzodiazepine, a barbiturate, a 5-HT2 antagonist, a dopaminergic drug or a combination thereof.
22. The method, use, or composition for use of any one of aspects 17-20, wherein the second therapeutic agent is adinazolam, allobarbital, alonimid, alprazolam, amantadine, amitriptyline, amobarbital, amoxapine, apomorphin, bentazepam, benzoctamine, benzhexol, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, co-beneldopa, co-careldopa, cyprazepam, desipramine, dexclamol, diazepam, dichloralphenazone, divalproex, diphenhydramine, doxepin, entacapone, estazolam, eszopiclone, ethchlorvynol, etomidate, fenobam, flunitrazepam, flurazepam, fluvoxamine, fluoxetine, fosazepam, gaboxadol, glutethimide, halazepam, hydroxyzine, imipramine, indiplon, levodopa, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, opicapone, oxazepam, paraldehyde, paroxetine, pentobarbital, perlapine, perphenazine, phenelzine, phenobarbital, pramipexole, prazepam, procyclidine, promethazine, propofol, protriptyline, quazepam, ramelteon, rasagiline, reclazepam, roletamide, ropinirole, rotigotine, safinamide, secobarbital, selegiline, sertraline, suproclone, temazepam, thioridazine, tracazolate, tranylcypromaine, trazodone, triazolam, trepipam, tricetamide, triclofos, trihexyphenidyl, trifluoperazine, trimetozine, trimipramine, uldazepam, valproate, venlafaxine, zaleplon, zolazepam, zolpidem, zopiclone, salts thereof, or a combination thereof.
23. The method, use, or composition for use of any one of aspects 1-22, wherein the formulation comprising an effective amount of piromelatine is administered in combination with a physical treatment method.
24. The method, use, or composition for use of aspect 23, wherein the physical treatment method comprises light therapy, psychiatric therapy, or a combination thereof.
25. The method, use, or composition for use of any one of aspects 1-24, wherein the subject is a human.
26. A method, use, or composition for use for treating a subject having parasomnias related to RBD comprising administering a formulation comprising about 2 mg to about 100 mg, about 5 mg to about 50 mg, or about 20 mg to about 40 mg of piromelatine to the subject per day.
27. The method, use, or composition for use of aspect 26, wherein the subject is diagnosed with RBD by video polysomnography (vPSG).
28. The method, use, or composition for use of aspect 27, wherein the vPSG measurement is quantified using a RBD Severity Scale (RBDSS).
29. The method, use, or composition for use of aspect 28, wherein the subject is diagnosed using a Clinical Global Impression-Improvement (CGI-I) scale.
30. The method, use, or composition for use of any one of aspects 26-29, wherein the subject is assessed using a dopamine transporter imaging—DaTscan, a blood testing, or a combination thereof.
31. The method, use, or composition for use of aspect 30, wherein the blood testing comprises measuring tumor necrosis factor-α (TNF-α) and/or C-reactive protein (CRP) levels in a serum sample of the subject.
32. The method, use, or composition for use of any one of aspects 26-31, wherein the subject is assessed using a Pittsburgh Sleep Quality Index (PSQI), an Epworth Sleepiness Scale (ESS), or a combination thereof.
33. The method of any one of aspects 26-32, wherein the formulation comprises one or more pharmaceutically acceptable diluents, preservatives, solubilizers, emulsifiers, adjuvants, excipients and/or carriers.
34. The method, use, or composition for use of any one of aspects 26-33, wherein the formulation is administered orally, parenterally, nasally, vaginally, rectally, sublingually, or topically.

### Other Embodiments

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims. Section headings, the materials, methods, and examples are illustrative only and not intended to be limiting.

Other aspects, advantages, and modifications are within the scope of the following claims.

1. A method for treating a subject having parasomnias related to Rapid Eye Movement (REM) sleep comprising administering a formulation comprising an effective amount of piromelatine to the subject.

2. The method of claim 1, wherein the subject has Rapid Eye Movement (REM) sleep behavior disorder (RBD).

3. The method of claim 1, wherein the subject has REM sleep without atonia (RSWA).

4. A method for treating a subject to attenuate disease progression into synucleinopathies or Parkinson's disease comprising administering a formulation comprising an effective amount of piromelatine to the subject.

5. A method of attenuating disease progression of parasomnias related to Rapid Eye Movement (REM) sleep into chronic post-traumatic stress disorder (PTSD) in a subject comprising administering a formulation comprising an effective amount of piromelatine to the subject.

6. The method of claim 1, wherein a non-REM sleep (NREM) low frequency band between 0.5 to 1.5 Hz is increased compared to baseline after administering piromelatine to the subject for about 4 weeks.

7. The method of claim 6, wherein a percentage ratio of an NREM low frequency band between 0.5 to 1.5 Hz is greater than 100% to about 120% or less compared to baseline after administering piromelatine to the subject for about 4 weeks.

8. The method of claim 2, wherein the effective amount of piromelatine is about 2 mg to about 100 mg per day, about 5 mg to about 50 mg per day, or about 20 mg to about 40 mg per day.

9. The method of claim 1, wherein the effective amount of piromelatine is about 50 mg per day.

10. The method of claim 1, wherein the formulation comprising an effective amount of piromelatine is administered orally, parenterally, nasally, vaginally, rectally, sublingually, or topically.

11. The method of claim 1, wherein the formulation comprising an effective amount of piromelatine is administered in a self-microemulsifying drug delivery system (SMEDDS).

12. The method of claim 1, wherein the formulation comprising an effective amount of piromelatine is administered in a unit dosage form.

13. The method of claim 1, wherein the formulation comprising an effective amount of piromelatine is administered once daily.

14. The method of claim 1, wherein the formulation comprising an effective amount of piromelatine is administered about 10 minutes to about 60 minutes prior to sleep time, or about 30 minutes prior to sleep time.

15. The method of claim 1, wherein the formulation comprising an effective amount of piromelatine is administered orally.

16. The method of claim 1, wherein the formulation comprising an effective amount of piromelatine is administered as a combination therapy with a second therapeutic agent.

17. The method of claim 16, wherein the combination therapy is administered concomitantly with the second therapeutic agent.

18. The method of claim 16, wherein the combination therapy comprises separately administering the formulation comprising an effective amount of piromelatine and one or more additional therapeutic agents.

19. The method of claim 16, wherein the combination therapy comprises an anti-inflammatory, an antioxidant, a hypnotic, an anxiolytic, an antipsychotic, an antianxiety agent, a minor tranquilizer, a melatonin agonist, a melatonin antagonist, melatonin, a benzodiazepine, a barbiturate, a 5-HT2 antagonist, a dopaminergic drug or a combination thereof.

20. The method of claim 16, wherein the second therapeutic agent is adinazolam, allobarbital, alonimid, alprazolam, amantadine, amitriptyline, amobarbital, amoxapine, apomorphin, bentazepam, benzoctamine, benzhexol, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, co-beneldopa, co-careldopa, cyprazepam, desipramine, dexclamol, diazepam, dichloralphenazone, divalproex, diphenhydramine, doxepin, entacapone, estazolam, eszopiclone, ethchlorvynol, etomidate, fenobam, flunitrazepam, flurazepam, fluvoxamine, fluoxetine, fosazepam, gaboxadol, glutethimide, halazepam, hydroxyzine, imipramine, indiplon, levodopa, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, opicapone, oxazepam, paraldehyde, paroxetine, pentobarbital, perlapine, perphenazine, phenelzine, phenobarbital, pramipexole, prazepam, procyclidine, promethazine, propofol, protriptyline, quazepam, ramelteon, rasagiline, reclazepam, roletamide, ropinirole, rotigotine, safinamide, secobarbital, selegiline, sertraline, suproclone, temazepam, thioridazine, tracazolate, tranylcypromaine, trazodone, triazolam, trepipam, tricetamide, triclofos, trihexyphenidyl, trifluoperazine, trimetozine, trimipramine, uldazepam, valproate, venlafaxine, zaleplon, zolazepam, zolpidem, zopiclone, salts thereof, or a combination thereof.

21. The method of claim 1, wherein the formulation comprising an effective amount of piromelatine is administered in combination with a physical treatment method.

22. The method of claim 21, wherein the physical treatment method comprises light therapy, psychiatric therapy, or a combination thereof.

23. The method of claim 8, wherein the subject is diagnosed with RBD by video polysomnography (vPSG).

24. The method of claim 23, wherein the vPSG measurement is quantified using a RBD Severity Scale (RBDSS).

25. The method of claim 24, wherein the subject is diagnosed using a Clinical Global Impression-Improvement (CGI-I) scale.

26. The method of claim 8, wherein the subject is assessed using a dopamine transporter imaging—DaTscan, a blood testing, or a combination thereof.

27. The method of claim 26, wherein the blood testing comprises measuring tumor necrosis factor-α (TNF-α) and/or C-reactive protein (CRP) levels in a serum sample of the subject.

28. The method of claim 8, wherein the subject is assessed using a Pittsburgh Sleep Quality Index (PSQI), an Epworth Sleepiness Scale (ESS), or a combination thereof.

29. The method of claim 8, wherein the formulation comprises one or more pharmaceutically acceptable diluents, preservatives, solubilizers, emulsifiers, adjuvants, excipients and/or carriers.
